(19) United States
(12) Patent Application Publication
(10) Pub. No.: US 2012/0050678 A1
(43) Pub. Date: Mar. 1, 2012

(54) PHOTOGRAPHING APPARATUS, AND CONTROL METHOD AND PROGRAM FOR THE SAME

(75) Inventor: Hiroshi Aoki, Saitama-shi (JP)

(73) Assignee: CANON KABUSHIKI KAISHA, Tokyo (JP)

(21) Appl. No.: 13/216,528

(22) Filed: Aug. 24, 2011

(30) Foreign Application Priority Data
Aug. 31, 2010 (JP) 2010-194669

(51) Int. Cl. A61B 3/14; A61B 3/117; A61B 3/12

(52) U.S. Cl. 351/206; 351/246

(57) ABSTRACT

Provided is a photographing apparatus capable of maintaining continuity of a series of operations of observation of an anterior ocular segment of an eye to be inspected, observation and photographing of a fundus thereof, and photographing of the anterior ocular segment. In the photographing apparatus, switching of a mode is performed among a first mode for forming on a photographing element (5) an image of an anterior ocular segment of an eye to be inspected (E), a second mode of performing at least one of observation and photographing of a fundus of the eye to be inspected (E), and a third mode of performing photographing of the anterior ocular segment. The photographing apparatus includes a focus lens control portion (28) for moving a focus lens (27) to a position which is set to correspond to the switched mode.

### BACKGROUND OF THE INVENTION

1. Field of the Invention

The present invention relates to a technology for a photographing apparatus provided with a mode for forming on a photographing unit an image of the anterior ocular segment of an eye to be inspected, a mode of performing at least one of observation and photographing of the fundus of the eye to be inspected, and a mode of performing photographing of the anterior ocular segment.

2. Description of the Related Art

A fundus camera, which is designed to observe and image the fundus of an eye to be inspected, is commonly used to perform fundus observation by means of a shared photographing optical system and perform anterior ocular alignment in a manner that an auxiliary lens, called anterior ocular lens, is inserted into the photographing optical system so that an image of the anterior ocular segment is formed on a photographing surface.

Japanese Patent Application Laid-Open No. H03-97437 discloses the configuration that makes it easier for an inspector to perform alignment of the anterior ocular segment of an eye to be inspected. In other words, the invention disclosed in Japanese Patent Application Laid-Open No. H03-97437 is configured to drive the focus lens so that an appropriate working distance may be obtained when the mode is switched from the fundus observation to the anterior ocular alignment. The invention is also configured to facilitate the fundus observation as well by returning the focus lens to the original position when the mode is switched to the fundus observation again after the switching from the fundus observation to the anterior ocular alignment.

The invention disclosed in Japanese Patent Application Laid-Open No. H03-97437 is very useful because the mode can be switched from the fundus observation to the anterior ocular alignment or from the anterior ocular alignment to the fundus observation easily with a simple configuration without the need for the inspector to operate the focus lens.

Meanwhile, to image the anterior ocular segment by the fundus camera, there is generally known a method of photographing the anterior ocular segment by inserting a diopter adjustment lens, moving the focus, and then moving the stage backward. Such fundus camera that consists of the fundus observing and photographing mode, the anterior ocular alignment mode, and the anterior ocular photographing mode puts less weight on the anterior ocular photographing mode. The inspector is thus required to operate the focus lens in the anterior ocular photographing mode. In addition, when the mode shifts from the anterior ocular photographing mode to the fundus observing and photographing mode, the position of the focus lens may be moved to a different position from the original one. The inspector therefore needs to adjust the focus lens again when the mode is returned to the fundus observing and photographing mode. This creates different focus operations among the modes, thus running the risk of confusing the inspector.

### SUMMARY OF THE INVENTION

It is therefore an object of the present invention to maintain continuity of a series of operations in a photographing apparatus provided with a mode for forming on a photographing unit an image of the anterior ocular segment of an eye to be inspected, a mode of performing at least one of observation and photographing of the fundus of the eye to be inspected, and a mode of performing photographing of the anterior ocular segment.

A photographing apparatus according to the present invention includes: a photographing unit; a switching unit for switching a mode among a first mode for forming on the photographing unit an image of an anterior ocular segment of an eye to be inspected, a second mode of performing at least one of observation and photographing of a fundus of the eye to be inspected, and a third mode of performing photographing of the anterior ocular segment; and a control unit for controlling movement of a focus lens on an optical axis between the eye to be inspected and the photographing unit, in which the control unit moves the focus lens to a position which is set corresponding to the mode switched by the switching unit.

According to the present invention, the continuity of a series of operations can be maintained in the photographing apparatus provided with the mode for forming on the photographing unit an image of the anterior ocular segment of the eye to be inspected, the mode of performing at least one of observation and photographing of the fundus of the eye to be inspected, and the mode of performing photographing of the anterior ocular segment.

Further features of the present invention will become apparent from the following description of exemplary embodiments with reference to the attached drawings.

### BRIEF DESCRIPTION OF THE DRAWINGS

FIG. 1 is a diagram illustrating a configuration of a fundus camera according to embodiments of the present invention.

FIG. 2 is a flowchart illustrating processing of the fundus camera according to the embodiments of the present invention.

FIG. 3 is a flowchart illustrating the processing of the fundus camera according to the embodiments of the present invention.

### DESCRIPTION OF THE EMBODIMENTS

Hereinafter, exemplary embodiments to which the present invention is applied are described in detail with reference to the accompanying drawings.

In the beginning, a first embodiment of the present invention is described. A fundus camera according to this embodiment is provided with a fundus observing and photographing mode, an anterior ocular alignment mode (also referred to as anterior ocular observing mode), and an anterior ocular photographing mode. The anterior ocular alignment mode is an application example of a first mode. The fundus observing and photographing mode is an application example of a second mode. The anterior ocular photographing mode is an application example of a third mode.

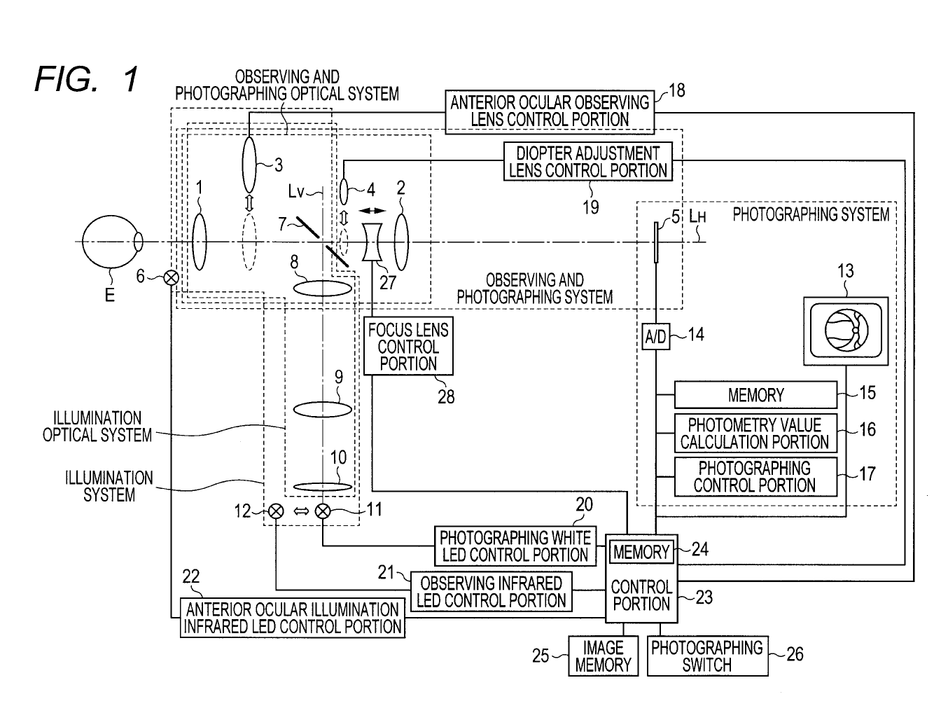

FIG. 1 is a diagram schematically illustrating a configuration of the fundus camera according to this embodiment.

In the fundus camera illustrated in FIG. 1, an objective lens 1 is disposed to oppose an eye to be inspected E. On an optical axis L_{H} of the objective lens 1, there are provided an image-forming lens 2, a focus lens 27 that is movable on the optical axis L_{H}, an anterior ocular observing lens 3 that is insertable and retreatable to and from the optical axis L_{H}, a diopter adjustment lens 4 that is similarly insertable and retreatable to and from the optical axis L_{H}, and a photographing element 5. In addition, at a substantially peripheral portion of the objective lens 1, an anterior ocular illumination infrared LED 6 for irradiating the anterior ocular segment of the eye to be inspected E is provided. Further, on the same optical axis L_{H}, an aperture mirror 7 is diagonally provided.

The components from the objective lens 1 to the image-forming lens 2 together constitute an observing and photographing optical system. The observing and photographing optical system and the photographing element 5 together constitute an observing and photographing system. On an optical axis L_{V} of the aperture mirror 7 in a reflection direction, a lens 8, a lens 9, and a condenser lens 10 are provided. In addition, on the same optical axis L_{V}, a photographing white LED 11 and an observing infrared LED 12 are provided so as to be switchable.

The components from the objective lens 1 to the condenser lens 10 together constitute an illumination optical system. The illumination optical system, the photographing white LED 11, and the observing infrared LED 12 together constitute an illumination system. The above-mentioned observing and photographing system and the illumination system together constitute a fundus camera optical portion. The fundus camera optical portion is placed on a slide table (not shown), enabling alignment with the eye to be inspected E.

An output of the photographing element 5 is converted into a digital signal by an A/D converter element 14. The digital signal is stored in a memory 15 and also connected to a photometry value calculation portion 16. The memory 15 and the photometry value calculation portion 16 are both connected to a control portion 23 for controlling the entire apparatus. The control portion 23 is connected to an image memory 25, and a still image imaged by the photographing element 5 is stored therein as digital image data.

The photographing element 5, the A/D converter element 14, the memory 15, and the photometry value calculation portion 16 as well as a monitor 13 for displaying an infrared observed image and a visible image which are imaged by the photographing element 5 and a photographing control portion 17 together constitute a photographing system. In addition, the photographing system is fixed to a housing of the fundus camera optical portion via a mount portion (not shown) in a detachable manner.

Note that, the anterior ocular observing lens 3, the diopter adjustment lens 4, and the focus lens 27 are moved under control of an anterior ocular observing lens control portion 18, a diopter adjustment lens control portion 19, and a focus lens control portion 28, respectively. Lighting of the anterior ocular illumination infrared LED 6 is controlled by an anterior ocular illumination infrared LED control portion 22. Lighting of the photographing white LED 11 and lighting of the observing infrared LED 12 are controlled by a photographing white LED control portion 20 and an observing infrared LED control portion 21, respectively. Further, switching between the photographing white LED 11 and the observing infrared LED 12 is controlled by the photographing white LED control portion 20 and the observing infrared LED control portion 21.

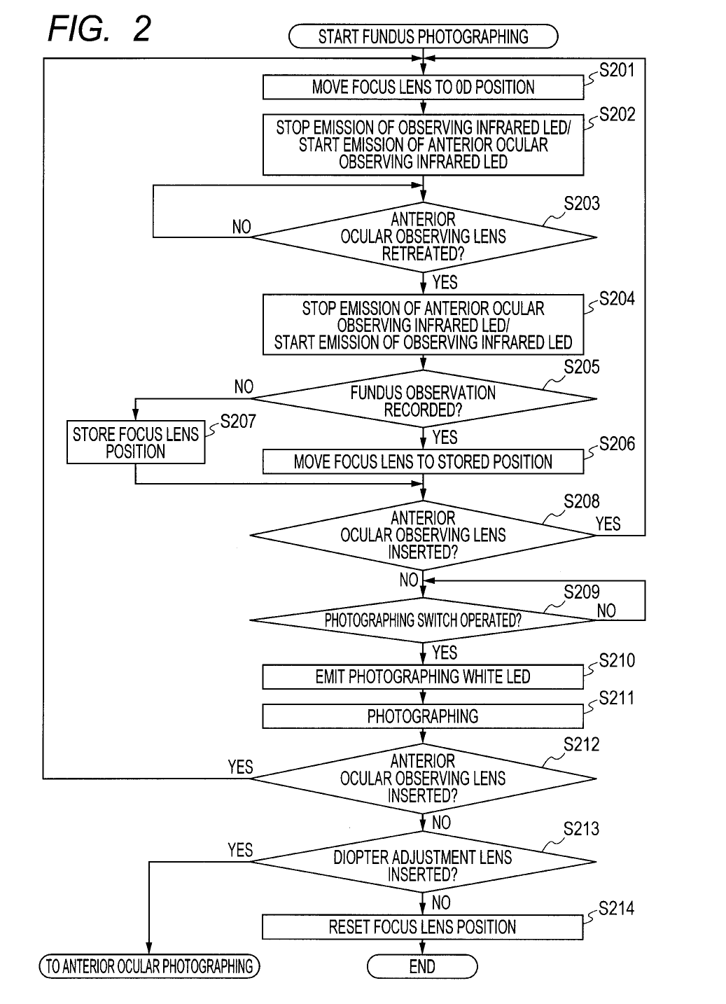

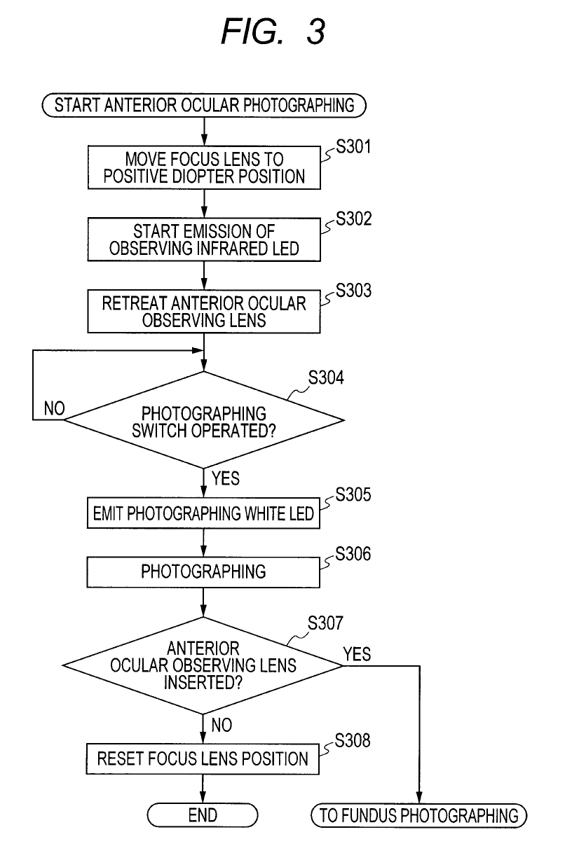

Next, processing of the fundus observing and photographing mode, the anterior ocular alignment mode, and the anterior ocular photographing mode performed by the fundus camera according to this embodiment is described with reference to FIG. 2 and FIG. 3. FIG. 2 and FIG. 3 are flowcharts illustrating the processing of the fundus camera according to this embodiment.

The inspector determines which of the anterior ocular photographing and the fundus photographing of the eye to be inspected E is performed. Described herein is the case where the inspector determines to perform the fundus photographing first. The inspector judges whether or not the anterior ocular observing lens 3 is inserted based on an observed image displayed on the monitor 13 or the like. When the anterior ocular observing lens 3 is not inserted onto the optical axis L_{H}, the inspector performs an operation of inserting the anterior ocular observing lens 3 onto the optical axis L_{H}. In response to the operation, the anterior ocular observing lens control portion 18 inserts the anterior ocular observing lens 3 onto the optical axis L_{H}. Then, the mode shifts to the anterior ocular alignment mode.

The focus lens control portion 28 moves the focus lens 27 to a zero-diopter (OD) position (Step S201). Next, the observing infrared LED control portion 21 stops emission of the observing infrared LED 12, and the anterior ocular illumination infrared LED control portion 22 starts emission of the anterior ocular illumination infrared LED 6 (Step S202).

In order to image the fundus of the eye to be inspected E, the inspector performs rough alignment between a center part of the eye to be inspected E and the optical axis L_{V}, to thereby form an image of the anterior ocular segment onto a photographing surface of the photographing element 5. The inspector judges whether or not the rough alignment has succeeded based on a captured image displayed on the monitor 13. The anterior ocular alignment mode is continued until the rough alignment succeeds. When the rough alignment succeeds, the inspector performs an operation of retreating the anterior ocular observing lens 3 from the optical axis L_{H}. In response to the operation, the anterior ocular observing lens control portion 18 retreats the anterior ocular observing lens 3 from the optical axis L_{H}. Then, the mode shifts from the anterior ocular alignment mode to the fundus observing and photographing mode.

The control portion 23 waits until the anterior ocular observing lens 3 is retreated from the optical axis L_{H} (Step S203). When the anterior ocular observing lens 3 is retreated from the optical axis L_{H}, the anterior ocular illumination infrared LED control portion 22 stops the emission of the anterior ocular illumination infrared LED 6, and the observing infrared LED control portion 21 starts the emission of the observing infrared LED 12 (Step S204).

The control portion 23 judges whether or not fundus observation history on the subject in question has been recorded. This judgment is performed, for example, in a manner that the control portion 23 judges whether or not a memory 24 has fundus observation history managed in association with identification information of the subject in question. When the fundus observation history on the subject in question has been recorded, the inspector performs an operation of moving the focus lens 27 to a position indicated by the fundus observation history. In response to the operation, the focus lens control portion 28 moves the focus lens 27 to that position (Step S206). On the other hand, when no fundus observation history on the subject in question has been recorded, that is, when it is the first time the fundus of the eye to be inspected E of the subject in question is observed, the inspector moves and positions the focus lens 27 finely while checking the fundus image on the monitor 13, and performs alignment. Then, when the position of the focus lens 27 is determined, the control portion 23 references information obtained from the focus lens control portion 28 and manages the position of the focus lens 27 in the memory 24 in association with the identification information of the subject in question as a fundus observation history (Step S207).

The inspector checks on the monitor 13 whether or not an index to be used in alignment is observed. The index is not observed when the fundus is greatly misaligned in the rough alignment or in other similar cases. If the index is not observed, the inspector performs again the operation of inserting the anterior ocular observing lens 3 onto the optical axis L_{H}. In response to the operation, the anterior ocular observing lens control portion 18 inserts the anterior ocular observing lens 3 onto the optical axis L_{H}.

The control portion 23 judges whether or not the anterior ocular observing lens 3 is inserted onto the optical axis L_{H} (Step S208). When the anterior ocular observing lens 3 is inserted onto the optical axis L_{H}, the mode returns to the anterior ocular alignment mode. In other words, the focus lens 27 is moved to the OD position (Step S201), and the processing is restarted from rough alignment. On the other hand, when the anterior ocular observing lens 3 is not inserted onto the optical axis L_{H}, the inspector checks on the monitor 13 whether or not the fundus is focused. When the inspector judges that the fundus is focused and photographing is ready, the inspector operates a photographing switch 26. Note that, when the fundus is not focused, the inspector moves the focus lens 27 while checking the fundus image on the monitor 13, and positions the focus lens 27. The association between the position determined in this operation and the identification information of the subject in question is overwritten as the fundus observation history.

The control portion 23 waits for an operation of the photographing switch 26 (Step S209). When the photographing switch 26 is operated, the photographing white LED control portion 20 causes the photographing white LED 11 to emit light (Step S210). The photographing control portion 17 executes photographing by using the photographing element 5 in synchronization with an emission timing of the photographing white LED 11 (Step S211).

The inspector judges whether or not to continue the fundus photographing. In the case of continuing the fundus photographing, the inspector performs an operation of inserting the anterior ocular observing lens 3 onto the optical axis L_{H}. In response to the operation, the anterior ocular observing lens control portion 18 inserts the anterior ocular observing lens 3 onto the optical axis L_{H}. The control portion 23 judges whether or not the anterior ocular observing lens 3 is inserted onto the optical axis L_{H} (Step S212). When the anterior ocular observing lens 3 is inserted onto the optical axis L_{H}, the mode shifts to the anterior ocular alignment mode, in which the focus lens 27 is moved to the OD position (Step S201), and the processing is restarted from rough alignment.

On the other hand, when the inspector judges to end the fundus photographing and perform anterior ocular photographing, the inspector performs an operation of inserting the diopter adjustment lens 4 onto the optical axis L_{H}. In response to the operation, the diopter adjustment lens control portion 19 inserts the diopter adjustment lens 4 onto the optical axis L_{H}. On the other hand, when the inspector judges not to perform the anterior ocular photographing and performs an end operation, the diopter adjustment lens 4 is not inserted onto the optical axis L_{H}.

The control portion 23 judges whether or not the diopter adjustment lens 4 is inserted onto the optical axis L_{H} (Step S213). When the inspector judges to perform the anterior ocular photographing and the diopter adjustment lens 4 is inserted onto the optical axis L_{H}, the mode shifts from the fundus observing and photographing mode to the anterior ocular photographing mode. On the other hand, when the inspector judges not to perform the anterior ocular photographing and the diopter adjustment lens 4 is not inserted onto the optical axis L_{H}, the control portion 23 resets the fundus observation history of the subject in question stored in the memory 24 (Step S214). This completes the examination.

Next, the anterior ocular photographing mode is described. The anterior ocular photographing mode is started when the inspector inserts the diopter adjustment lens 4 onto the optical axis L_{H}.

When the control portion 23 detects that the diopter adjustment lens 4 is inserted onto the optical axis L_{H}, the focus lens control portion 28 moves the focus lens 27 to a positive diopter position (Step S301). The observing infrared LED control portion 21 starts the emission of the observing infrared LED 12 (Step S302). The anterior ocular observing lens control portion 18 retreats the anterior ocular observing lens 3 from the optical axis L_{H} (Step S303).

The inspector performs alignment to move the optical axis L_{H} to near the center of the position of the anterior ocular segment where the inspector intends to image. The alignment is continued until the alignment succeeds. When the alignment succeeds, the inspector operates the photographing switch 26. The control portion 23 waits for the operation of the photographing switch 26 (Step S304). When the photographing switch 26 is operated, the photographing white LED control portion 20 causes the photographing white LED 11 to emit light (Step S305). The photographing control portion 17 executes photographing by using the photographing element 5 in synchronization with an emission timing of the photographing white LED 11 (Step S306).

The inspector judges whether or not to continue the anterior ocular photographing. In the case of continuing the anterior ocular photographing, the inspector performs re-alignment to perform photographing again. On the other hand, when the inspector judges to end the anterior ocular photographing, the inspector judges whether or not to perform fundus photographing. In the case of not performing the fundus photographing, the inspector performs an end operation. In response to the end operation, the control portion 23 resets the fundus observation history of the subject in question stored in the memory 24 (Step S308). This completes the examination. On the other hand, when the inspector judges to perform the fundus photographing, the inspector performs an operation of inserting the anterior ocular observing lens 3 onto the optical axis L_{H}. In response to the operation, the anterior ocular observing lens control portion 18 inserts the anterior ocular observing lens 3 onto the optical axis L_{H}. Then, the mode shifts from the anterior ocular photographing mode to the anterior ocular alignment mode.

As described above, in this embodiment, optimum positions of the focus lens are set to correspond to the anterior ocular alignment mode, the fundus observing and photographing mode, and the anterior ocular photographing mode, and when the mode shifts to the respective modes, the focus lens is moved to the corresponding position. It is therefore possible to maintain continuity of a series of operations in the fundus camera provided with the anterior ocular alignment mode, the fundus observing and photographing mode, and the anterior ocular photographing mode.

Next, a second embodiment of the present invention is described. Note that, a fundus camera according to the second embodiment has the same configuration as that illustrated in FIG. 1. Hereinafter, the difference in processing between the first embodiment and the second embodiment is described.

The second embodiment enables the movement of the focus lens 27 in the anterior ocular photographing mode. For example, when the focus lens 27 is moved in the anterior ocular photographing mode from the positive diopter position to another position, the position of the focus lens 27 in the anterior ocular photographing mode is reset to the another position from the positive diopter position. Then, when the mode returns to the anterior ocular photographing mode again from another mode, the focus lens 27 is moved to the reset position.

Next, a third embodiment of the present invention is described. Note that, a fundus camera according to the third embodiment has the same configuration as that illustrated in FIG. 1. Hereinafter, the difference in processing between the first embodiment and the third embodiment is described.

In the first embodiment, alignment of each of the right and left eyes to be inspected is enabled by a slide mechanism. In the third embodiment, on the other hand, the slide mechanism includes a detection unit for detecting switching between the right and left eyes to be inspected, and the positions of the focus lens 27 corresponding to the right and left eyes to be inspected at the time of the fundus observing and photographing mode are stored in advance. When the mode returns to the fundus observing and photographing mode again, at the time of switching between the right and left eyes to be inspected, the focus lens 27 is moved to the position which is stored to correspond to a detection result of the detection unit and each of the modes.

Further, the present invention is also implemented by executing the following processing. Specifically, in this processing, software (program) for implementing the functions of the above-mentioned embodiments is supplied to a system or an apparatus via a network or various kinds of storage medium, and a computer (or CPU, MPU, etc.) of the system or the apparatus reads and executes the program.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-194669, filed Aug. 31, 2010, which is hereby incorporated by reference herein in its entirety.

What is claimed is:

1. A photographing apparatus, comprising:
a photographing unit;
a switching unit for switching a mode among a first mode for forming on the photographing unit an image of an anterior ocular segment of an eye to be inspected, a second mode of performing at least one of observation and photographing of a fundus of the eye to be inspected, and a third mode of performing photographing of the anterior ocular segment; and
a control unit for controlling movement of a focus lens on an optical axis between the eye to be inspected and the photographing unit,
wherein the control unit moves the focus lens to a position which is set to correspond to the mode switched by the switching unit.

2. A photographing apparatus according to claim 1, wherein, in the first mode, the control unit moves the focus lens to a zero-diopter position at a time of the at least one of the observation and the photographing of the fundus.

3. A photographing apparatus according to claim 1, wherein, in the third mode, the control unit moves the focus lens to a positive diopter position at a time of the at least one of the observation and the photographing of the fundus.

4. A photographing apparatus according to claim 1, further comprising a setting unit for resetting, when the focus lens is moved in one of the second mode and the third mode from a position which is set to correspond to the mode in question to another position, the another position as a position of the focus lens, the position corresponding to the mode in question.

5. A photographing apparatus according to claim 1, further comprising a detection unit for detecting switching between right and left eyes to be inspected,
wherein the control unit moves the focus lens to a position which is set to correspond to a detection result of the detection unit and the mode switched by the switching unit.

6. An ophthalmologic apparatus, comprising:
a focusing unit which is movable on an optical axis;
a switching unit for switching between an anterior ocular observing mode of observing an anterior ocular segment of an eye to be inspected and an anterior ocular photographing mode of photographing the anterior ocular segment of the eye to be inspected; and
a control unit for moving the focusing unit to a position on the optical axis, the position corresponding to the mode switched by the switching unit.

7. A method of controlling an ophthalmologic apparatus, comprising:
switching between an anterior ocular observing mode of observing an anterior ocular segment of an eye to be inspected and an anterior ocular photographing mode of photographing the anterior ocular segment of the eye to be inspected; and
moving a focusing unit to a position on an optical axis, the position corresponding to the mode switched in the switching.

8. A recording medium having recorded thereon a program for causing a computer to execute the steps of the method of controlling an ophthalmologic apparatus according to claim 7.
